SEQUENCE LISTING (13 sequences)

SEQ ID NO: 1 - RNA, 22 nt, Artificial Sequence
  Synthetic primer
auguagggcuaaaagccauggg

SEQ ID NO: 2 - RNA, 19 nt, Artificial Sequence
  Synthetic primer
aagcagcugccucugaggc

SEQ ID NO: 3 - RNA, 23 nt, Artificial Sequence
  Synthetic primer
aucccuugcaggggcuguugggu

SEQ ID NO: 4 - RNA, 21 nt, Artificial Sequence
  Synthetic primer
ugaguuggccaucugagugag

SEQ ID NO: 5 - RNA, 23 nt, Artificial Sequence
  Synthetic primer
uuaaugcuaaucgugauaggggu

SEQ ID NO: 6 - RNA, 22 nt, Artificial Sequence
  Synthetic primer
augcugacauauuuacuagagg

SEQ ID NO: 7 - RNA, 23 nt, Artificial Sequence
  Synthetic primer
ugugcuugcucgucccgcccgca

SEQ ID NO: 8 - RNA, 22 nt, Artificial Sequence
  Synthetic primer
uuuguucguucggcucgcguga

SEQ ID NO: 9 - RNA, 21 nt, Artificial Sequence
  Synthetic primer
caaagcgcuucccuuuggagc

SEQ ID NO: 10 - RNA, 23 nt, Artificial Sequence
  Synthetic primer
gaacgcgcuucccuauagagggu

SEQ ID NO: 11 - RNA, 23 nt, Artificial Sequence
  Synthetic primer
aucgcugcgguugcgagcgcugu

SEQ ID NO: 12 - RNA, 27 nt, Artificial Sequence
  Synthetic primer
cacuguaggugauggugagagugggca

SEQ ID NO: 13 - RNA, 23 nt, Artificial Sequence
  Synthetic primer
uaaaucccauggugccuucuccu

(19) United States
(12) Patent Application Publication
(10) Pub. No.: US 2014/0274923 A1
(43) Pub. Date: Sep. 18, 2014

(54) MIRNAS AS A PROGNOSTIC BIOMARKER IN PEDIATRIC HEART FAILURE

(71) Applicant: The Regents of the University of Colorado, a body corporate, Denver, CO (US)

(72) Inventors: Carmen Sucharov, Superior, CO (US); Brian Stauffer, Golden, CO (US); Shelley Miyamoto, Parker, CO (US)

(21) Appl. No.: 14/203,022

(22) Filed: Mar. 10, 2014

Related U.S. Application Data
(60) Provisional application No. 61/782,140, filed on Mar. 14, 2013.

(51) Int. Cl. C12Q 1/68

(52) U.S. Cl. CPC C12Q 1/6883; C12Q 2600/112; C12Q 2600/178
USPC 514/26; 506/9; 435/6.12

(57) ABSTRACT

The present invention provides for methods of predicting progression of pediatric heart failure using miRNA biomarkers, as well as subsequent treatment of patients based on their classification.

This application claims benefit of priority to U.S. Provisional Application Ser. No. 61/782,140, filed Mar. 14, 2013, the entire contents of which are hereby incorporated by reference.

Pursuant to 37 C.F.R. 1.821(c), a sequence listing is submitted herewith as an ASCII compliant text file named “UTECP0036US_ST25.txt”, created on Mar. 7, 2014 and having a size of ˜3 KB. The content of the aforementioned file is hereby incorporated by reference in its entirety.

### BACKGROUND OF THE INVENTION

1. Field of the Invention

The present invention relates generally to the fields of pathology and pediatric medicine. More particularly, it concerns classification of various forms of pediatric heart failure using miRNA biomarkers.

2. Description of Related Art

Heart Failure (HF) in children remains poorly understood with most clinical treatment paradigms extrapolated solely from experience in adults. Emerging experimental evidence and epidemiologic data suggest that the pediatric HF population is distinctly different from adult patients (1). As in adults, the etiology of HF in children is heterogeneous. The most common cause of HF and reason for cardiac transplantation in children older than 1 year is dilated cardiomyopathy (DCM) (2). The incidence of DCM is about 5.5 per 100,000 adults per year and 0.34 to 1.09 per 100,000 children per year (3-7). While DCM is less common in children than in adults, the clinical consequences are similarly devastating with a 5-year rate of death or transplantation of 40-60% (6, 8). However, while there has been marked improvement in adult HF outcomes secondary to advances in diagnostic and therapeutic options, there has been no improvement in transplant-free survival for children with DCM over the past 3 decades (9). In addition, although many children with DCM who present in acute decompensated HF have a poor outcome, 15-35% of children have recovery of ventricular function (8, 10) This relatively high potential for recovery and the limited (although improving) graft survival in pediatric heart transplant recipients make risk stratification of children presenting with DCM of paramount importance (2). To date, a reliable risk stratification profile to predict outcome for children with DCM does not exist. This problem hinders the ability of pediatric cardiologists to confidently determine the most appropriate medical treatment plan for these patients, which contributes to suboptimal outcomes. As such, there is a significant need for improved prognostic methods that allow for the accurate classification of pediatric patients who will proceed to end stage HF versus those who will recover spontaneously.

### SUMMARY OF THE INVENTION

Thus, in accordance with the present invention, there is provided a method of identifying a pediatric subject having dilated cardiomyopathy (DCM) that will recover without heart transplant comprising (a) obtaining an miRNA-containing sample from said patient; (b) measuring the levels of one or more miRNAs selected from the group consisting of hsa-miR-135b#/*, hsa-miR-646, hsa-miR-623, hsa-miR-571, hsa-miR-155, hsa-miR-628-5p, hsa-miR636 and hsa-miR-375; and (c) identifying said patient as one that will recover without heart transplant when one or more of hsa-miR-135b#/*, hsa-miR-646, hsa-miR-623, and hsa-miR-571 are increased as compared levels from a subject requiring heart transplant, and/or wherein one or more of hsa-miR-155, hsa-miR-628-5p, hsa-miR636 and hsa-miR-375 are decreased as compared levels from a subject requiring heart transplant. The levels for 2, 3, 4, 5, 6, 7 or all 8 of said miRNAs may be measured. When said patient is identified as recovering without heart transplant, said method further comprises treating said patient with pharmacologic therapy or mechanical circulatory support. When said patient is identified as not recovering without transplant, said method further comprises monitoring said patient for progression to non-stable heart failure. When said patient is not identified as not recovering, said method further comprises performing heart transplant.

The method may further comprise comprising determining whether said recovering patient will improve or will have stable/persistent DCM. The measuring may comprise microarray hybridization and/or RT-PCR. The sample may be blood, serum, urine or plasma. The patient may be diagnosed with idiopathic DCM. The patient may be diagnosed with myocarditis. The method may further comprise performing a control analysis that examines one or more of the levels for hsa-miR-16, hsa-miR-573 hsa-miR-197 and/or hsa-miR-106b. The method may further comprising measuring one or more miRNAs selected from the group consisting of hsa-miR-518d, hsa-miR-639, hsa-miR-1183, hsa-miR-605 and hsa-miR-523 and further identifying said patient as one that will recover without heart transplant when one or more of hsa-miR-518d, hsa-miR-639, hsa-miR-1183 and hsa-miR-605 are increased as compared levels from subject requiring heart transplant, and/or wherein hsa-miR-523 is decreased as compared levels from a subject requiring heart transplant. The levels for 3, 4, 5, 6, 7, 8, 9, 10, 11, 12 or all 13 of said analytical miRNAs may be measured.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. miRNA expression profile in left ventricular heart tissue of pediatric and adult idiopathic (non-ischemic) DCM patients. 6 non-failing (NF) adults, 6 adults with DCM (failing), 5 NF children and 5 children with DCM were analyzed. Only miRNAs with a p-value of <0.05 are shown. Red: up-regulated miRNAs; green: down-regulated miRNAs.

FIGS. 2A-D. Volcano plot showing comparisons between circulating miRNA expression of different groups. P-value is represented in a −log₁₀ scale. miRNAs that have a p-value of <0.05 are shown above the blue line (−log₁₀ value=1.301). miRNAs represented in green color are down-regulated, in red color are up-regulated, and in black color are <2 fold regulated (up or down). (FIG. 2A) Comparison between Group 1 (patient samples obtained pre-transplant or on the day of transplant) and non-failing subjects; (FIG. 2B) comparison between Group 1 and DCM patients that recovered; (FIG. 2C) comparison between DCM patients that recovered and non-failing subjects; (FIG. 2D) comparison between patient samples drawn pre-transplant (including those patients that eventually died) and samples drawn on the day of transplant.

FIGS. 3A-D. Venn diagrams of comparisons between various groups. (FIG. 3A) Comparison between miRNAs up-regulated in Group 1 non-failing (NF), recovery and mechanical circulatory support (MCS) patients; (FIG. 3B) comparisons between miRNAs down-regulated in Group 1, NF, recovery and MCS patients; (FIG. 3C) comparisons between miRNAs up-regulated in Group 1, NF and recovery patients; (FIG. 3D) comparisons between miRNAs down-regulated in Group 1, NF and recovery patients. Comparison details are described in the figure.

FIGS. 4A-B. Comparison of changes in serum and heart miRNA in DCM and non-failing patients. (FIG. 4A) miRNAs up-regulated in the heart of DCM patients when compared to non-failing controls were down-regulated in serum. (FIG. 4B) miRNAs that were unchanged in the heart were also unchanged in serum.

FIG. 5. Random Forest (RF) Analysis of heart failure group (pre-transplant, transplant or death) compared to recovery group—normalized to hsa-miR-16 and hsa-miR-573. RF analysis was performed for hsa-miRs-135b#, 646, 623 571 normalized to hsa-miR-573, and hsa-miRs-155, 628-5p, 636 and 375 normalized to hsa-miR-16. Results show sample stratification based on heart failure outcome (recovery versus transplant or death).

FIG. 6. Receiver Operator Curve (ROC) to determine sensitivity and specificity of selected miRNAs as biomarkers—based on normalization to hsa-miR-16 and hsa-miR-573. ROC was performed based on RF results shown in FIG. 5. Results show sensitivity and specificity of the analyzed miRNAs to predict heart failure outcome (recovery versus transplant or death).

FIG. 7. Random Forest Analysis of heart failure group (pre-transplant, transplant or death) compared to recovery group—normalized to hsa-miR-106b and hsa-miR-573. RF analysis was performed for hsa-miRs-135b#, 646, 623 571 normalized to hsa-miR-573, and hsa-miRs-155, 628-5p, 636 and 375 normalized to hsa-miR-106b. Results show sample stratification based on heart failure outcome (recovery versus transplant or death).

FIG. 8. Receiver Operator Curve (ROC) to determine sensitivity and specificity of selected miRNAs as biomarkers—based on normalization to hsa-miR-106b and hsa-miR-573. ROC was performed based on RF results shown in FIG. 7. Results show sensitivity and specificity of the analyzed miRNAs to predict heart failure outcome (recovery versus transplant or death).

FIGS. 9A-D. Boxplots for miRNAs confirmed by RT-PCR. Expression of miR-155 (FIG. 9A), miR-646 (FIG. 9B), miR-623 (FIG. 9C) and miR-636 (FIG. 9D) was analyzed for the following groups: recovered, pre-transplant, pre-transplant including the 4 patients that died, day of transplant sample (transplant) and day of transplant including the 8 patients that were analyzed only by RT-PCR.

FIG. 10A-F. ROC for any one (FIG. 10A), two (FIG. 10B) or three (FIG. 10C) miRNAs that were different between recovered and pre-transplant groups (including patients that died) and ROC for any one (FIG. 10D), two (FIG. 10E) or three (FIG. 10F) miRNAs that were different between recovered and day of transplant samples (including the 8 patients analyzed only by RT-PCR).

### DETAILED DESCRIPTION OF ILLUSTRATIVE EMBODIMENTS

A reliable risk stratification profile for children with HF does not exist. Also, there are a limited number of studies of biomarkers in children with heart failure, and there has not been a previous investigation of circulating miRNAs in any pediatric cardiovascular population. The inventors' previous studies demonstrated obvious age-related differences in the expression of miRNAs in heart tissue of adults and children with DCM, and the results described here now show marked differences in the expression of circulating miRNAs in pediatric end-stage DCM patients compared to those that recover heart function. Based on these data, it is clear that extrapolation of adult circulating miRNA data is not appropriate, and that the age-related miRNA differences described underscore the importance of performing these studies in the pediatric population.

The present application has several features that are designed to address historical limitations in understanding HF in children. First and primarily, children are a “vulnerable” population, without the ability to provide informed consent (Park and Grayson, 2008). It is therefore very difficult to perform the more invasive mechanistic studies that have been so fruitful in understanding HF pathophysiology in adults. Unfortunately, this ethical dilemma is recognized as a significant impediment to major advances in pediatric evidence-based medicine in other diseases as well (Park and Grayson, 2008). The current proposal proposes a minimal risk type of analysis. Second, HF in children is inherently heterogeneous, including congenital heart disease to cardiomyopathies of various forms. It is for this reason that the current proposal has narrowed the focus to the most common cause of HF and indication for transplant in children, DCM. Taken together, the information presented here is the first evidence specific to circulating miRNA in children with DCM and provides a basis for using miRNA profiles to categorize pediatric DCM/HF populations. These and other aspects of the invention are described below.

### I. PEDIATRIC HEART FAILURE

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Approximately half of the DCM cases are idiopathic, with the remainder being associated with known disease processes. For example, serious myocardial damage can result from certain drugs used in cancer chemotherapy (e.g., doxorubicin and daunoribucin). In addition, many adult DCM patients are chronic alcoholics. Fortunately, for these patients, the progression of myocardial dysfunction may be stopped or reversed if alcohol consumption is reduced or stopped early in the course of disease. Peripartum cardiomyopathy is another form of DCM, as is disease associated with infectious sequelae. In sum, cardiomyopathies, including idiopathic DCM, are significant public health problems. In the United States, approximately half a million individuals are diagnosed with heart failure each year, with a mortality rate approaching 50%. The cost to diagnose, treat and support patients suffering from these diseases is well into the billions of dollars.

The etiology of heart failure in children is different from that in adults; therefore, it is erroneous to assume that response to clinical therapy in children will be identical to that demonstrated in adults. Similar to adults, idiopathic dilated cardiomyopathy is one of the most prevalent causes of heart failure in children. However, the causes of DCM in children are different from adults, with a much higher incidence of infectious myocarditis, familial and genetic diseases. In contrast to the adult population, there is virtually no ischemic heart disease in children and a very high prevalence of congenital heart disease in the heart failure population. It is important to note that due to the anatomical heterogeneity of the pediatric heart failure population, references to ventricular function usually specify systemic versus non-systemic ventricle instead of references to right and left ventricular function as in the adult population. Although the annual incidence of cardiomyopathies in children is lower than in adults (1 per 100,000 in children vs 4-5 per 100,000 individuals in adults), the severity of disease is equally devastating, with 1- and 5-year rates of death or transplantation of 30% and 40% respectively.

MicroRNAs (miRNA, miRs) are small noncoding ˜22 nucleotide (nt) RNAs capable of modulating the expression of many genes (Ambros 2001). Several studies have analyzed the expression of miRNAs in the heart, and recently have shown that miRNAs can also be useful biomarkers. These studies have shown that expression of a specific miRNA can be correlated with disease etiology and is associated with adverse outcomes in adult patients with DCM. More recently, studies have shown the importance and applicability of blood circulating miRNAs in the early diagnosis of a myocardial infarction (Fichtlscherer et al., 2010) and in the correlation with outcomes in animal models of heart failure (Montgomery et al., 2011). The inventors have characterized miRNA profiles in children with DCM and have determined their effectiveness in predicting clinical outcomes in this population. These results suggest that circulating miRNAs are useful biomarkers in pediatric DCM patients, and are proposed as for use in risk stratification to classify outcomes in the high risk pediatric HF population. These and other aspects of the invention are described below.

### II. miRNAs

miRNAs are small noncoding ˜22 nucleotide (nt) RNAs capable of modulating the expression of many genes (Ambros 2001) with estimates suggesting that as much as 60% of the genome is subject to their regulation (Friedman et al., 2009). At the most simplistic level, miRNAs bind to target mRNAs by recognition of reverse complementary 6-8 nucleotide “seed” sequences, most frequently located within 3′ untranslated regions (3′UTR). miRNAs can cause translational repression or RNA destabilization (Filipowicz et al., 2008 and Filipowicz et al., 2005).

Over the past few years, several array-based studies have been published detailing changes in miRNA expression in normal versus failing adult human heart (Van Rooij et al., 2006, Sucharov et al., 2008, Ikeda et al., 2007, Naga et al., 2009, Matkovich et al., 2009 and Thum et al., 2007). The results of these studies have been nicely summarized in recent reviews (Condorelli et al., 2010 and Small et al., 2010). Thus far, the number of miRs detected by arrays in the heart (above statistical threshold) is on the order of ˜150-200 miRs. As summarized by Condorelli et al. (Cordorelli et al., 2010), ˜89 miRs were found to be up-regulated, ˜45 miRs were down-regulated, and ˜29 miRs were unchanged in expression. miRs that repeatedly surface as dynamically regulated (up or down) in adult heart failure include several let-7s, miR-1, miR-133a/b, miR-19a/b, miR-150, miR-195, miR-199, miR-221, miR-23a/b, miR-29a/b, miR-30 family, and miR-320.

In the past 3 years, over 300 manuscripts have been published that showed the effectiveness of circulating miRNAs as reliable biomarkers. Circulating miRNAs have been shown to be stable in the blood and can be useful diagnostic biomarkers in a broad range of diseases in adults including cancer, liver disease, autoimmune diseases, and cardiovascular diseases (Kerr et al, 2011, Dai and Ahmed 2011, Farazi et al., 2011 and Kukreja et al., 2011), and more recently in pediatric Crohn's disease (Zahm et al., 2011). Recent work indicates that miRNA signatures can, in adult patients, specifically correlate with myocardial infarction (MI), viral myocarditis (Corsten et al., 2010), heart failure (Tijsen et al, 2011), and can differentiate between myocardial infarction and unstable angina (Rothman et al., 2011), suggesting that they can be a powerful diagnostic biomarker to stratify adult patients with heart disease. In addition, miR-1 and miR-133 are rapidly released in the circulation after an MI (156 minutes post MI), display a higher peak than TnI, and can be effective biomarkers for the early diagnosis of MI (Fichtlscherer et al., 2010). Further, Satoh et al. (Satoh et al., 2010), have recently demonstrated that increased expression of miR-208 in endomyocardial biopsies appears to be associated with adverse outcomes in adults with dilated cardiomyopathy. Recently, Montgomery et al. showed that therapeutic inhibition of miR-208a in hypertension-induced heart failure in rats improved cardiac function and survival (Montgomery et al., 2011). Interestingly, they also showed that expression of the circulating miRNAs, miR-499 and miR-423-5p correlated with improvements in cardiac function. These studies strongly suggest that circulating miRNAs can be powerful biomarkers of heart disease. Several of these studies investigating miRNAs as biomarkers for adult cardiovascular disease analyzed target expression of miRNAs known to change in heart tissue. Interestingly, the only study that analyzed circulating miRNA expression based on miRNA array showed differential expression of several miRNAs that are unknown to change in heart tissue (Tijsen et al., 2010). This work demonstrates the importance of array-based studies on the initial characterization of circulating miRNA expression and its correlation with heart failure etiology, outcomes and response to treatment.

Expression of circulating miRNAs has not been analyzed in pediatric heart failure patients. A recent study showed age-related differences in the expression of circulating miRNAs (Olivieri et al., 2012). In this study, differential expression of a subset of miRNAs in healthy twenty-year-old subjects was compared to healthy octogenarian and centenarian groups. Importantly, miRNAs whose expression is significantly higher in the centenarian group (miR-200b, miR-200c, miR-212, miR-425 and miR-579) are either not expressed or expressed at very low levels in pediatric non-failing control subjects examined here (data not shown). Furthermore, the results presented here show that the miRNA expression profile in the ventricles and blood of children with DCM is unique, with little overlap to the adult HF population. These results highlight the inapplicability of using an adult miRNA profiles in pediatric HF patients, and underscore the importance of defining a uniquely pediatric signature of circulating miRNAs in this population.

Expression of circulating miRNAs was determined at the time of presentation with DCM. The various classifications that are possible include (i) end stage HF (defined as listing for transplant or treatment with MCS; (ii) recovery of ventricular function; or (iii) stable/persistent DCM.

A. miRNAs Modulated in Pediatric HF Patients

hsa-miR-135b#/*.
(SEQ ID NO: 1)
Sequence is 5′-AUGUAGGGCUAAAAGCCAUGGG-3′.
hsa-miR-646.
(SEQ ID NO: 2)
Sequence is 5′-AAGCAGCUGCCUCUGAGGC-3′.
hsa-miR-623.
(SEQ ID NO: 3)
Sequence is 5′-AUCCCUUGCAGGGGCUGUUGGGU′3′.
hsa-miR-571.
(SEQ ID NO: 4)
Sequence is 5′-UGAGUUGGCCAUCUGAGUGAG-3′.

hsa-miR-155.

miR-155 is a microRNA that in humans is encoded by the MIR155 host gene or MIR155HG. miR-155 plays an important role in various physiological and pathological processes. Exogenous molecular control in vivo of miR-155 expression may inhibit malignant growth, viral infections, and attenuate the progression of cardiovascular diseases. The MIR155HG was initially identified as a gene that was transcriptionally activated by promoter insertion at a common retroviral integration site in B-cell lymphomas and was formerly called BIC (B-cell Integration Cluster). It was latter found that the MIR155HG was composed of three exons that span a 13 kb region within human chromosome 21 (Hsa21 band q21.3). The MIR155HG is transcribed by RNA polymerase II and the resulting ˜1,500 nucleotide RNA is capped and polyadenylated. The 23 nucleotide single-stranded miR-155, which is harbored in exon 3, is subsequently processed from the parent RNA molecule. Sequence is 5′-UUAAUGCUAAUCGUGAUAGGGGU-3′ (SEQ ID NO: 5).

hsa-miR-628-5p.
(SEQ ID NO: 6)
Sequence is 5′-AUGCUGACAUAUUUACUAGAGG-3′.

hsa-miR 636.

miR-636 has been identified as one of three key miRNAs associated with the anti-ageing myelodysplastic syndromes (MDS). Its levels correspond to high discrimination between MDS and normal controls, and expression is decreased in MDS. In this way it can be used as a potential diagnostic marker for MDS. Resistance to glucocorticoids (GC) used in the treatment of blood-related malignancies greatly impairs their clinical utility. The active glucocorticoid receptor GR-α is required for an effective response to GCs, but this is significantly downregulated in GC-resistant cell lines MM.1Re and MM.1RL. miR-636 has been found to be differentially expressed between GC-sensitive and GC-resistant MM.1 cell lines. It has therefore been identified as a possible candidate responsible for postranscriptional silencing of GR-α in GC-resistant cells. Sequence is 5′-UGUGCUUGCUCGUCCCGCCCGCA-3′ (SEQ ID NO: 7).

hsa-miR-375.

miR-375 is specifically expressed in the pancreatic islets and brain. miR-375 was one of the first miRNAs identified in the pancreas (Poy et al, 2004), and remains one of the best characterised in terms of function. It is expressed in the pancreas and pituitary gland, organs linked by their role in hormone secretion, and expression levels increase during pancreas organogenesis. Loss-of-function studies showed that miR-375 is essential for β-cell formation in zebrafish (Kloosterman et al, 2007). miR-375 knockout mice have decreased numbers of β-cells and increased numbers of α-cells. The increase in glucagon levels, combined with the reduction in insulin levels, results in hyperglycaemia. This shows the importance of miR-375 in the establishment of normal pancreatic cell mass through the targeting of a group of genes which control cellular growth and proliferation in the developing pancreas. Further evidence for the involvement of miR-375 in pancreas development includes the fact that its expression is regulated by several transcription factors important in pancreatic development and function, including HNF6, INSM1, NGN3, NEUROD1, and PDX-1. Sequence is 5′-UUUGUUCGUUCGGCUCGCGUGA-3′ (SEQ ID NO: 8).

hsa-miR-518d.
(SEQ ID NO: 9)
Sequence is 5′-CAAAGCGCUUCCCUUUGGAGC-3′.
hsa-miR-523.
(SEQ ID NO: 10)
Sequence is 5′-GAACGCGCUUCCCUAUAGAGGGU-3′.
hsa-miR-639.
(SEQ ID NO: 11)
Sequence is 5′-AUCGCUGCGGUUGCGAGCGCUGU-3′.
hsa-miR-1183.
(SEQ ID NO: 12)
Sequence is 5′-CACUGUAGGUGAUGGUGAGAGUGGGCA-3′.
hsa-miR-605.
(SEQ ID NO: 13)
Sequence is 5′-UAAAUCCCAUGGUGCCUUCUCCU-3′.

B. Methods of Measuring miRNA Levels

With regard to assessing the expression of miRNAs from a subject having or suspected of having DCM, any method of detection known to one of skill in the art falls within the general scope of the present invention. In general, nucleic acids are detected using homologous nucleic acid segments as probes or primers in nucleic acid hybridization. Commerically available systems, such as Qiagen's miScript System™ are available for detection of miRNAs. Various aspects of nucleic acid detection as discussed below.

1. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

2. In Situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g., plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

3. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific miRNA species isolated from a cell. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Chip Technologies and Arrays

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of an miRNA with respect to diagnostic, as well as preventative and treatment methods of the invention.

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### III. METHODS OF TREATING HEART FAILURE

A. Therapeutic Regimens

Current medical management of cardiac failure in the setting of a cardiovascular disorder includes the use of at least two types of drugs: inhibitors of the renin-angiotensin system, and β-adrenergic blocking agents (Eichhorn and Bristow, 1996 and Bristow, 1999). Other pharmaceutical agents that have been disclosed for treatment of heart failure include angiotensin II receptor antagonists (U.S. Pat. No. 5,604,251) and neuropeptide Y antagonists (WO 98/33791). Despite currently available pharmaceutical compounds, prevention and treatment of cardiac hypertrophy, and subsequent heart failure, continue to present a therapeutic challenge.

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids). Surgical intervention, such as pacemakers, bypass and transplant may also be contemplated. Treatment regimens depend on the clinical situation, and determining those patients that will self-resolve, versus those who will progress to full blown HF, verus those who will have stable/persistent disease is critical to applying the correct treatments to the patients.

B. Pharmacologic Therapy

One may provide to the patient more “standard” pharmaceutical cardiac therapies. Examples of other therapies include, without limitation, anti-hypertensives, cardiotonics, anti-thrombotics, vasodilators, hormone antagonists, inotropes, diuretics, endothelin antagonists, calcium channel blockers, phosphodiesterase inhibitors, ACE inhibitors, angiotensin receptor type 2 antagonists and cytokine blockers/inhibitors, and HDAC inhibitors. A particular form of combination therapy will include the use of β2-selective agonists. This combination is designed to take advantage of the differential β-adrenergic receptor expression changes unique to pediatric heart failure subjects.

Combinations may be achieved by contacting cardiac cells with (i.e., administering to patients) a single composition or pharmacological formulation that includes both agents, or by contacting the cell with two distinct compositions or formulations, at the same time, wherein one composition includes the expression construct and the other includes the agent. Alternatively, the therapy using a β2-selective agonist may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either a β2-selective agonist, or the other agent will be desired. In this regard, various combinations may be employed. By way of illustration, where the β2-selective agonist is “A” and the other agent is “B,” the following permutations based on 3 and 4 total administrations are exemplary:

A/B/A B/A/B B/B/A A/A/B B/A/A A/B/B B/B/B/A B/B/A/B

A/A/B/B A/B/A/B A/B/B/A B/B/A/A B/A/B/A B/A/A/B B/B/B/A

A/A/A/B B/A/A/A A/B/A/A A/A/B/A A/B/B/B B/A/B/B B/B/A/B

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof. Other combinations are likewise contemplated. Some specific agents are described below.

1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol), simvastatin (zocor), atorvastatin (Lipitor) or rosuvastatin (crestor).

d. Nicotinic Acid Derivatives

Non-limiting examples of nicotinic acid derivatives include nicotinate, acepimox, niceritrol, nicoclonate, nicomol and oxiniacic acid.

e. Thyroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantine), heparin, sulfinpyranone (anturane), ticlopidine (ticlid), clopidigrel (Plavix) and ticagrelor (Brilinta).

c. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemorrhage or an increased likelihood of hemorrhaging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamin K.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrythmic agents (sodium channel blockers), Class II antiarrythmic agents (beta-adrenergic blockers), Class II antiarrythmic agents (repolarization prolonging drugs), Class IV antiarrhythmic agents (calcium channel blockers) and miscellaneous antiarrythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocamide (tonocard) and mexiletine (mexitil). Non-limiting examples of Class IC antiarrhythmic agents include encamide (enkaid) and flecamide (tambocor).

b. Repolarization Prolonging Agents

Non-limiting examples of an agent that prolong repolarization, also known as a Class III antiarrhythmic agent, include amiodarone (cordarone) and sotalol (betapace).

c. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a miscellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

d. Miscellaneous Antiarrhythmic Agents

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an α-adrenergic blocker or an α-adrenergic antagonist, include, amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives.

Non-limiting examples of arylethanolamine derivatives include amosulalol, bufuralol, dilevalol, labetalol, pronethalol, sotalol and sulfinalol.

Benzothiadiazine Derivatives.

Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives.

Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives.

Non-limiting examples of dihydropyridine derivatives include amlodipine, felodipine, isradipine, nicardipine, nifedipine, nilvadipine, nisoldipine and nitrendipine.

Guanidine Derivatives.

Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines.

Non-limiting examples of hydrazines/phthalazines include budralazine, cadralazine, dihydralazine, endralazine, hydracarbazine, hydralazine, pheniprazine, pildralazine and todralazine.

Imidazole Derivatives.

Non-limiting examples of imidazole derivatives include clonidine, lofexidine, phentolamine, tiamenidine and tolonidine.

Quanternary Ammonium Compounds.

Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives.

Non-limiting examples of reserpine derivatives include bietaserpine, deserpidine, rescinnamine, reserpine and syrosingopine.

Suflonamide Derivatives.

Non-limiting examples of sulfonamide derivatives include ambuside, clopamide, furosemide, indapamide, quinethazone, tripamide and xipamide.

g. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

7. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine administration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furtherene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

c. Inotropic Agents

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

C. Surgical Therapeutic Agents

Such surgical therapies for cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise extracorporeal membrane oxygenation (ECMO), an intra-aortic balloon counterpulsation, left (or bi-) ventricular assist device or combination thereof.

In cases of severe HF, heart transplant is a treatment of last resort. Approximately 350-400 pediatric heart transplantation procedures are performed worldwide each year, representing about 10% of the total number of heart transplants performed. Congenital malformations are still the most common indication for infant heart transplantation, occurring in approximately 1 in 10,000 live births. The most common indication for heart transplantation in older children is cardiomyopathy. The number of children who have failing cardiac function late after palliative surgery for congenital heart disease is increasing. Survival in excess of 20 years after pediatric heart transplantation has been achieved. Most programs now report that more than 70% of their recipients survive at least 5 years.

The donor supply remains inadequate. Improved public and physician awareness of donor issues is the most important factor in increasing donor supply because many potential donors are not identified as such. Thus, the ability to identify patients who do not actually need transplant, i.e., those that will recover spontaneously, is critical to providing effective care to patients who need transplants.

After heart transplantation, the major health concern is rejection of the new heart. Medications are provided to suppress the child's immune system (and thus prevent rejection), but at the same time put the child at risk for infection and other side effects. A biopsy procedure allows for inspection of the heart muscle for signs of rejection. The majority of children who receive new hearts resume a relatively normal and age-appropriate lifestyle.

A variety of post-transplant risks exist. For example, where there is concomitant disease of other organ systems, patients may require additional support. Irreversible pulmonary, renal, hepatic, or systemic disease, coexisting neoplasm, insulin-dependent diabetes mellitus with end-organ damage, and active peptic ulcer disease or diverticulosis have been considered traditional contraindications to heart transplantation. A need for renal dialysis at the time of transplantation has been identified as a risk factor for survival. Also, the nephrotoxicity of immunosuppressant medication and the risk of progressive renal dysfunction after transplantation means that patients with irreversible moderate to severe renal dysfunction and end-stage heart disease are increasingly treated with combined heart-kidney transplantation. Similar strategies of combined heart-liver transplantation have been used in the presence of irreversible hepatic dysfunction.

Seropositivity for hepatitis B virus surface antigen before transplantation is frequently associated with clinical liver disease after transplantation. Heart transplant recipients with preoperative hepatitis C infection have also been found to be at risk for the development of potentially fatal liver disease. Human immunodeficiency virus (HIV) infection has been a contraindication to heart transplantation owing to very poor outcomes for heart transplantation in HIV-positive patients in the past.

Pediatric heart disease is associated with a myriad of mechanisms that may increase pulmonary artery pressures and pulmonary vascular resistance, such as left atrial hypertension due to systemic ventricular dysfunction, anatomic obstruction to pulmonary venous return, pulmonary veno-occlusive disease, pulmonary arteriolar constriction, anatomic obstruction of the large pulmonary arteries, increased pulmonary blood flow from congenital heart disease with left-to-right shunting, and accessory sources of pulmonary blood flow from aortopulmonary collaterals. One or all of these mechanisms may occur in any given patient with pediatric heart disease. Discontinuity or severe obstruction within large pulmonary arteries can lead to differing pulmonary pressure and vascular resistance in one portion of a patient's pulmonary bed compared with another. Assessment of pulmonary vascular resistance in a patient with pediatric heart disease considered for heart transplantation is critical because of the well-established risk of postoperative heart failure and mortality in patients undergoing heart transplantation with high pulmonary vascular resistance.

Several psychosocial variables have been considered absolute or relative contraindications to transplantation in adults, such as use of illicit drugs, alcohol abuse, mental retardation, and documented medical noncompliance. Substance abuse, noncompliance, and psychological problems have been associated with increased morbidity and mortality after heart transplantation in adults.

D. Drug Formulations and Routes for Administration to Patients

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs stable and allow for uptake by target cells. Aqueous compositions of the present invention comprise an effective amount of the drug, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route. This includes is administered orally, nasally, bucally, transdermally, in sustained release, in controlled release, in delayed release, as a suppository, sublingually, intravenously, subcutaneously, or intraosseously. Alternatively, administration may be by intradermal, intramuscular, or intraperitoneal, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

For oral administration the polypeptides of the present invention generally may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. DEFINITIONS

As used herein, the term “heart failure” is broadly used to mean any condition that reduces the ability of the heart to pump blood. As a result, congestion and edema develop in the tissues. Many factors may result in heart failure, including reduced coronary blood flow, damage to the heart valves, congenital abnormalities, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses modalities aimed at improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, heart size, left ventricular internal dimension, heart rate, etc.), as well as any effect upon the animal's survival. In use of animal models, the response of treated animals and untreated animals is compared using any of the assays described herein. A compound which causes an improvement in any parameter associated with heart failure used in the screening methods of the instant invention may thereby be identified as a therapeutic compound.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “agonist” refers to molecules or compounds which mimic the action of a “native” or “natural” compound. Agonists may be homologous to these natural compounds in respect to conformation, charge or other characteristics. Thus, agonists may be recognized by receptors expressed on cell surfaces. This recognition may result in physiologic and/or biochemical changes within the cell, such that the cell reacts to the presence of the agonist in the same manner as if the natural compound was present. Agonists may include proteins, nucleic acids, carbohydrates, or any other molecules that interact with a molecule, receptor, and/or pathway of interest.

As used herein, the terms “antagonist” and “inhibitor” refer to molecules, compounds, or nucleic acids which inhibit the action of a cellular factor that may be involved in cardiac hypertrophy or failure. Antagonists may or may not be homologous to these natural compounds in respect to conformation, charge or other characteristics. Thus, antagonists may be recognized by the same or different receptors that are recognized by an agonist. Antagonists may have allosteric effects which prevent the action of an agonist. Antagonists and inhibitors may include proteins, nucleic acids, carbohydrates, or any other molecules which bind or interact with a receptor, molecule, and/or pathway of interest.

As used herein, the term “modulate” refers to a change or an alteration in a biological activity. Modulation may be an increase or a decrease in protein activity, a change in kinase activity, a change in binding characteristics, or any other change in the biological, functional, or immunological properties associated with the activity of a protein or other structure of interest. The term “modulator” refers to any molecule or compound which is capable of changing or altering biological activity as described above.

As used herein, “pediatric” means that the subject is less than 18 years of age.

### V. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

microRNA Extraction.

miRNA is extracted using isolated serum mixed with LS-Trizol and extracted with Quiagen miRNeasy kit according to manufacturer's recommendation. Integrity of the RNA is determined by the University of Colorado microarray core. The Agilent 2100 Bioanalyzer with an RNA LabChip Kit is used to evaluate RNA integrity.

Array Analysis.

miRNA array is performed using the ABI technology. This technology allows the identification of 765 miRNAs from plasma or serum. Arrays are performed according to manufacturer's recommendation. Briefly, total RNA is reverse transcribed using a pool of primers specific for each miRNA. To account for miRNAs expressed at very low levels, cDNA products are pre-amplified using specific primers. Real-time PCR reactions are performed in 384 well plates containing sequence-specific primers and TaqMan probes in the ABI7900. Two 384 well plates are used for each sample to detect miRNAs and appropriate controls.

### Example 2

### Results

miRNA Expression in Pediatric Heart Failure.

The inventors' previous study showed several changes in miRNA expression in adult failing hearts (Sucharov et al. 2008). These changes were similar to published results from other groups (Condorelli et al. 2010 and Small et al. 2010). The inventors subsequently utilized their Pediatric Heart Tissue Bank to determine miRNA expression in the left ventricles of pediatric patients transplanted secondary to DCM (FIG. 1). These results were subsequently confirmed by RT-PCR in 12 non-failing and 24 DCM patients (data not shown). Seventeen miRNAs were significantly regulated in pediatric failing hearts; the majority of those are antithetically regulated or not regulated in adults (analysis done based on previous published work shown in FIG. 1 and (Sucharov et al. 2008) and results summarized in (Condorelli et al. 2010 and Small et al. 2010).

miR-130b, miR-204, miR-331-3p, miR-223, miR-188-5p, miR-1281, miR-572, miR-765 and miR-1268 are not regulated in adults with HF. Three miRNA (miR-638, miR-7 and miR-132) are antithetically regulated in pediatric and adult failing hearts. Directionality of expression of miR-27a/b, miR-125a and miR-486 is the same in both populations although they are not detected in all studies. Importantly, expression of several miRNAs known to change in adults with cardiac disease (miR-133, miR-29, miR-1, miR-20, miR-30, miR-23, miR-34 (for a complete list see (Condorelli et al. 2010 and Small et al. 2010) do not change in pediatric failing hearts. Although the biological consequences of these differences are presently unknown, it is likely that there is a dramatic difference in the regulation of downstream miRNA targets in pediatric failing hearts. These results underscore the differences in miRNA expression profile in the hearts of adult and pediatric patients with DCM and emphasize the importance of characterizing molecular differences in this population.

miRNAs as a Circulating Biomarker in Pediatric DCM Patients.

To determine the feasibility of miRNAs as circulating biomarkers in pediatric DCM patients, the inventors analyzed serum miRNA expression in a cohort of normal/non-failing children and a cohort of children with DCM. Array analysis was performed in using the Applied Biosystems (ABI) technology allowing for the identification of 765 miRNAs from plasma or serum. miRNA probes are distributed in two 384-well plates, and the hsa-miRs-573, 16, 106b and 197 were used as the endogenous control. Analysis of expression of these miRNAs in these patients showed no difference in their expression, validating their function as an endogenous control (data not shown). The results shown in FIGS. 2A-D represent analysis performed in one array plate, which contains approximately 380 miRNAs. Out of those, 60 miRNAs were consistently expressed in all samples. The analyzed serum was obtained from 11 non-failing children (<18 years old, 60% male) (NF) and 34 patients with DCM. Of the 34 patients with DCM, 25 patients underwent transplant (which included familial, IDC, arrhythmia and viral myocarditis), 4 died and 5 recovered (ventricular function normalized). For the 34 DCM patients, 23 samples were obtained at the time of pre-transplant evaluation or on the day of transplant (this group is referred to as Group 1 in the figures and includes the 4 patients that died prior to transplant), 6 samples were obtained from the patients with DCM that eventually recovered (blood drawn when the patient was in HF), and 12 samples were obtained from DCM patients following initiation of MCS. As shown in FIGS. 2A-D, comparison between the different groups shows that several miRNAs are specific for disease outcome. Interestingly, and in support of the specificity of the data, no differences were observed in the circulating miRNA profile of samples drawn at the time of pre-transplant evaluation (includes those that ultimately died) compared to the samples drawn on the day of transplant (FIG. 2D). Therefore, as described previously, these groups were combined for further analysis (Group 1). The median age of the DCM cohort was 4.7 years, 56% were female and the average time between pre-transplant blood draw and transplant was 90 days. Details regarding the 5 patients that recovered are outlined in the Table 1 (EF—ejection fraction with normal being ≧50%; FS—shortening fraction with normal being ≧30%).

TABLE 1

| | | | | | Time (yrs) | |
|---|---|---|---|---|---|---|
| | Age | | | | from | |
| | (yrs) | | | | presen- | |
| | at | | | EF or FS | tation | |
| | presen- | | | at time | to | EF at |
| Patient | tation | Sex | DX | of miRNA | recovery | recovery |
| 1 | 1.4 | F | Myocarditis | EF 24% | 0.9 | 63% |
| 2 | 1.2 | F | Myocarditis | EF 17% | 2.0 | 70% |
| 3 | 10.6 | M | Myocarditis | FS 8% | 1.1 | FS 35% |
| 4 | 14 | F | Arrhythmia | EF 21% | 0.3 | 58% |
| 5 | 0.9 | F | Myocarditis | EF 18% | 0 9 | 60% |

Further comparisons show that some miRNAs may be specifically used as biomarkers. More specifically, as shown in FIG. 3A, two miRNAs (miR-193a-5p and miR-29a) are consistently and significantly up-regulated in Group 1 when compared to NF or recovered group. Furthermore, one miRNA (miR-342-3p) is significantly up-regulated in Group 1 when compared to NF, recovered or the post-MCS groups. Similarly, miR-181c is down-regulated in Group 1 when compared to NF, recovered or the post-MCS groups (FIG. 3B). These miRNAs may be related to recovery or unloading and may be a prognostic marker for pediatric DCM patients. In addition, although several miRNAs are up-regulated in Group 1 when compared to NF (FIG. 3C), only three miRNAs are up-regulated in the recovered group when compared to NF, and only one (miR-345) is commonly up-regulated between Group 1 and the recovery group. For miRNAs that are down-regulated in Group 1 and recovery compared to NF, 26 miRNAs are uniquely down-regulated in the recovery group (FIG. 3D). More specifically, and as shown in FIGS. 5 and 7, hsa-miRNAs-13b#, 646, 623, 571 and 375 are consistently up-regulated in patients that recovered from HF when compared to Group 1. Similarly, hsa-miRNAs-155, 628-5p and 636 are consistently down-regulated in patients that recovered from heart failure when compared to Group 1. FIGS. 6 and 8 show receiver operator curve graphs that define the specificity and sensitivity of these miRNAs as biomarkers to determine HF recovery in pediatric patients. These results show that at the time they were suffering from HF secondary to DCM, children that eventually recover display a very unique circulating miRNA profile. This miRNA profile thus represents a biomarker signature of miRNAs that are specific to those children with DCM that have the potential to recover. The miRNA expression profile for those that recover was very similar regardless of the underlying cause of DCM (see Table 1) and may thus be associated with potential for recovery and not disease etiology. Although myocarditis was prevalent in patients that recovered, some patients listed for transplant were also diagnosed with myocarditis, and the miRNA expression profile segregated with outcomes and not disease etiology. However, these data reflect a very small number of patients, and needs to be validated in a larger dataset.

Circulating and Heart Tissue miRNA Expression.

To determine if changes in miRNA expression in DCM patients were similar in heart and blood, a sub-analysis of miRNAs expressed in the left ventricle and serum from non-failing and end-stage DCM patients was performed. Array results presented in FIGS. 2A-D were confirmed by RT-PCR in 12 non-failing and 24 DCM patients, and expression of miRNAs analyzed by RT-PCR was investigated in serum. As shown in FIGS. 4A-B, of the 16 miRNAs analyzed, 5 are not expressed in serum, 4 are up-regulated in the heart of DCM patients but down-regulated in the blood (FIG. 4A), and 7 are unchanged in serum and heart (FIG. 4B). These results suggest a relationship between expression of miRNAs in DCM in heart and blood.

In order to confirm the array data, RT-PCR was performed. In addition to the samples used for arrays, 8 patients with samples collected only on the day of transplant were used in the RT-PCR reaction. Since this group was not used in the array experiments its analysis is important to confirm the results obtained with the original sample set. Out of 9 miRNAs that differentiated the recovered group from the transplanted group, 4 miRNAs (miR-623, miR-636, miR155 and miR-646) showed a strong correlation between RT-PCR and array (data not shown). As shown in FIGS. 9A-D, box plot analysis showed similar results between the different groups with the exception of miR-623 which is different from recovery only when compared to day of transplant. The addition of the 8 patient samples did not affect the means of day of transplant except for a small increase in the mean of miR-646. These results suggest that these miRNAs can predict the need for transplant in pediatric IDC patients.

In order to determine the ability of these miRNAs to accurately predict the need for transplant, ROC was performed. The inventors analyzed ROC for the presence of any one miRNA that was different in the pre-transplant or transplant groups when compared to the recovered group. As shown in FIGS. 10A-F, the presence of any one miRNA different than recovered for pre-transplant patients resulted in an AUC of 0.833 and for transplant patients (including the added 8 patients) was 0.929. FIGS. 10A-F also shows AUC for any two or three miRNAs. These results suggest that these miRNAs are highly predictive of the need for transplant in the pediatric IDC population.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
Ambros, Cell 107(7):823-6, 2001.
Bellus, J. Macromol. Sci. Pure Appl. Chem., A31(1): 1355-1376, 1994.
Bentin and Nielsen, Biochemistry, 35:8863-8869, 1996.
Bentwich et al., Nat. Genet., 37:766-770, 2005.
Bristow, Cardiology, 92:3-6, 1999.
Condorelli et al., Eur Heart J., 31(6):649-58, 2010
Corsten et al., Circ Cardiovasc Genet., 3(6):499-506, 2010.
Dai et al., Transl Res., 157(4):163-79, 2011.
Durand et al., Ann. Med., 27:311-317, 1995.
Eichhorn and Bristow, Circulation, 94:2285-2296, 1996.
European Appln. 320 308
European Appln. 329 822
European Appln. 373 203
European Appln. 785 280
European Appln. 799 897
Farazi et al., J Pathol., 223(2):102-15. PMCID: 3069496, 2011.
Fichtlscherer et al., Circ Res., 107(5):677-84, 2010.
Filipowicz et al., Current Opinion in Structural Biology, 15(3):331-41, 2005.
Filipowicz et al., Nature Reviews Genetics, 9(2):102-14, 2008.
Fodor et al., Science, 251:767-773, 1991.
Frohman, In: PCR Protocols: A Guide To Methods And Applications, Academic Press, N.Y., 1990.
GB Appln. 2 202 328
Hacia et al., Nature Genet., 14:441-449, 1996.
Ikeda et al., Physiol Genomics, 31(3):367-73, 2007.
Innis et al., Proc. Natl. Acad. Sci. USA, 85(24):9436-9440, 1988.
Kamb et al., Nat. Genet., 8(1):23-26, 1994.
Kamb et al., Science, 2674:436-440, 1994.
Kashani-Sabet et al., Antisense Res. Dev., 2:3, 1992.
Kaur et al., Biochemistry, 45(23):7347-7355, 2006.
Kerr et al., Transl Res., 157(4):241-52., PMCID: 3063952, 2011.
Kukreja et al., Mol Pharmacol., 2011
Kwoh et al., Proc. Natl. Acad. Sci. USA, 86:1173, 1989.
Matkovich et al., Circulation, 119(9):1263-71, 2009.
Montgomery et al., Circulation 2011
Naga et al., J Biol Chem., 284(40):27487-99, 2009.
Nobori et al., Nature (London), 368:753-756, 1995.
Ohara et al., Proc. Natl. Acad. Sci. USA, 86:5673-5677, 1989.
Park and Grayson, J Allergy Clin Immunol., 121(5):1103-7, 2008.
PCT Appln. PCT/US87/00880
PCT Appln. PCT/US89/01025
PCT Appln. WO 00/53722
PCT Appln. WO 01/38580
PCT Appln. WO 01/68255
PCT Appln. WO 03/020898
PCT Appln. WO 03/022421
PCT Appln. WO 03/023058
PCT Appln. WO 03/029485
PCT Appln. WO 03/040410
PCT Appln. WO 03/053586
PCT Appln. WO 03/066906
PCT Appln. WO 03/067217
PCT Appln. WO 03/076928
PCT Appln. WO 03/087297
PCT Appln. WO 03/091426
PCT Appln. WO 03/093810
PCT Appln. WO 03/100012
PCT Appln. WO 03/100448A1
PCT Appln. WO 04/020085
PCT Appln. WO 04/027093
PCT Appln. WO 05/021570
PCT Appln. WO 05/115481
PCT Appln. WO 05/121371
PCT Appln. WO 09/923,256
PCT Appln. WO 09/936,760
PCT Appln. WO 88/10315
PCT Appln. WO 89/06700
PCT Appln. WO 90/07641
PCT Appln. WO 93/17126
PCT Appln. WO 93/23569
PCT Appln. WO 94/02595
PCT Appln. WO 94/02595
PCT Appln. WO 94/14226
PCT Appln. WO 95/11995
PCT Appln. WO 95/21265
PCT Appln. WO 95/21944
PCT Appln. WO 95/35505
PCT Appln. WO 96/10390
PCT Appln. WO 96/10391
PCT Appln. WO 96/10392
PCT Appln. WO 96/31622
PCT Appln. WO 97/10365
PCT Appln. WO 97/27317
PCT Appln. WO 97/43450
PCT Appln. WO 98/33791
PCT Appln. WO 98/39352
PCT Appln. WO 99/14226
PCT Appln. WO 99/35505
Pease et al., Proc. Natl. Acad. Sci. USA, 91:5022-5026, 1994.
Poy et al., Nature, 432:226-230, 2004.
Remington's Pharmaceutical Sciences, 15th Ed., 1035-1038 and 1570-1580, 1990.
Rothman et al., Heart, 97(20):v-e7, 2011.
Sambrook and Russell, Molecular Cloning: A Laboratory Manual 3rd Ed., Cold Spring Harbor Laboratory Press, 2001.
Sambrook et al., In: Molecular cloning: a laboratory manual, 2nd Ed., Cold Spring Harbor Laboratory Press, Cold Spring Harbor, N.Y., 1989.
Satoh et al., J. Card Fail., 16(5):404-10, 2010.
Shoemaker et al., Nature Genetics, 14:450-456, 1996.
Small et al., Circulation, 121:1022-1032, 2010.
Soutschek et al., Nature, 432(7014):173-178, 2004.
Stockmans et al., Curr. Opin. Oncol., 20:614-620, 2008.
Sucharov et al., J. Mol. Cell Cardiol., 45:185-192, 2008.
Thum et al., Circulation, 116(3):258-67, 2007.
Tijsen et al., Circ Res., 106(6):1035-9, 2010.
U.S. Pat. No. 4,683,195
U.S. Pat. No. 4,683,202
U.S. Pat. No. 4,800,159
U.S. Pat. No. 4,879,236
U.S. Pat. No. 4,883,750
U.S. Pat. No. 4,981,957
U.S. Pat. No. 5,118,800
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,319,080
U.S. Pat. No. 5,324,633
U.S. Pat. No. 5,359,044
U.S. Pat. No. 5,384,261
U.S. Pat. No. 5,393,878
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,514,785
U.S. Pat. No. 5,519,134
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,567,811
U.S. Pat. No. 5,571,639
U.S. Pat. No. 5,576,427
U.S. Pat. No. 5,580,726
U.S. Pat. No. 5,580,732
U.S. Pat. No. 5,591,722
U.S. Pat. No. 5,593,839
U.S. Pat. No. 5,597,909
U.S. Pat. No. 5,599,672
U.S. Pat. No. 5,599,695
U.S. Pat. No. 5,604,251
U.S. Pat. No. 5,610,300
U.S. Pat. No. 5,610,287
U.S. Pat. No. 5,624,711
U.S. Pat. No. 5,627,053
U.S. Pat. No. 5,631,134
U.S. Pat. No. 5,639,603
U.S. Pat. No. 5,639,873
U.S. Pat. No. 5,646,265
U.S. Pat. No. 5,654,413
U.S. Pat. No. 5,658,734
U.S. Pat. No. 5,658,873
U.S. Pat. No. 5,661,028
U.S. Pat. No. 5,665,547
U.S. Pat. No. 5,667,972
U.S. Pat. No. 5,670,633
U.S. Pat. No. 5,695,940
U.S. Pat. No. 5,700,637
U.S. Pat. No. 5,700,920
U.S. Pat. No. 5,739,169
U.S. Pat. No. 5,744,305
U.S. Pat. No. 5,792,747
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,871,986
U.S. Pat. No. 5,876,932
U.S. Pat. No. 5,882,864
U.S. Pat. No. 5,900,481
U.S. Pat. No. 5,902,880
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,146,886
U.S. Pat. No. 6,268,490
U.S. Pat. No. 6,368,799
U.S. Pat. No. 6,383,749
U.S. Pat. No. 6,617,112
U.S. Pat. No. 6,638,717
U.S. Pat. No. 6,720,138
U.S. Patent Pubin. 2008/0009439
UK Appln. 8 803 000
Van Rooij, et al., Proc Natl Acad Sci USA, 103(48):18255-60, 2006.
Wahlestedt et al., Proc. Natl. Acad. Sci. USA, 97:5633-5638, 2000
Walker et al., Proc. Natl. Acad. Sci. USA, 89:392-396, 1992.
Zahm et al., J Pediatr Gastroenterol Nutr., 53(1):26-33, 2011.

What is claimed is:

1. A method of identifying a pediatric subject having dilated cardiomyopathy (DCM) that will recover without heart transplant comprising:
(a) obtaining an miRNA-containing sample from said patient;
(b) measuring the levels of one or more miRNAs selected from the group consisting of hsa-miR-135b#/*, hsa-miR-646, hsa-miR-623, hsa-miR-571, hsa-miR-155, hsa-miR-628-5p, hsa-miR636 and hsa-miR-375; and
(c) identifying said patient as one that will recover without heart transplant when one or more of hsa-miR-135b#/*, hsa-miR-646, hsa-miR-623, and hsa-miR-571 are increased as compared levels from a subject requiring heart transplant, and/or wherein one or more of hsa-miR-155, hsa-miR-628-5p, hsa-miR636 and hsa-miR-375 are decreased as compared levels from a subject requiring heart transplant.

2. The method of claim 1, wherein when said patient is identified as recovering without heart transplant, said method further comprises treating said patient with pharmacologic therapy or mechanical circulatory support.

3. The method of claim 1, wherein when said patient is identified as not recovering without transplant, said method further comprises monitoring said patient for progression to non-stable heart failure.

4. The method of claim 1, wherein when said patient is not identified as not recovering, said method further comprises performing heart transplant.

5. The method of claim 1, wherein levels for 2, 3, 4, 5, 6, 7 or all 8 of said miRNAs are measured.

6. The method of claim 1, further comprising determining whether said recovering patient will improve or will have stable/persistent DCM.

7. The method of claim 1, wherein measuring comprises microarray hybridization.

8. The method of claim 1, wherein measuring comprises RT-PCR.

9. The method of claim 1, wherein said sample is blood, serum or plasma.

10. The method of claim 1, wherein said patient is diagnosed with idiopathic DCM.

11. The method of claim 1, wherein said patient is diagnosed with myocarditis.

12. The method of claim 1, further comprising performing a control analysis that examines one or more of the levels for hsa-miR-16, hsa-miR-573 hsa-miR-197 and/or hsa-miR-106b.

13. The method of claim 1, further comprising measuring one or more miRNAs selected from the group consisting of hsa-miR-518d, hsa-miR-639, hsa-miR-1183, hsa-miR-605 and hsa-miR-523 and further identifying said patient as one that will recover without heart transplant when one or more of hsa-miR-518d, hsa-miR-639, hsa-miR-1183 and hsa-miR-605 are increased as compared levels from subject requiring heart transplant, and/or wherein hsa-miR-523 is decreased as compared levels from a subject requiring heart transplant.

14. The method of claim 13, wherein levels for 3, 4, 5, 6, 7, 8, 9, 10, 11, 12 or all 13 of said miRNAs are measured.
